(19) United States
(12) Patent Application Publication
(10) Pub. No.: US 2020/0390795 A1
(43) Pub. Date: Dec. 17, 2020

(54) DIETARY SUPPLEMENT COMPOSITIONS FOR TREATING HYPERTENSION AND METHODS OF USING SAME FOR TREATING HYPERTENSION

(71) Applicant: AMERICAN BLUETEC COMPANY, COMMERCE TOWNSHIP, MI (US)

(72) Inventor: Dexian DOU, COMMERCE TOWNSHIP, MI (US)

(21) Appl. No.: 16/988,246

(22) Filed: Aug. 7, 2020

Related U.S. Application Data
(63) Continuation-in-part of application No. 16/439,201, filed on Jun. 12, 2019.

(51) Int. Cl. A61K 31/7064; A61K 31/065; A61P 3/06; A23L 33/105

(52) U.S. Cl. CPC A61K 31/7064; A61K 31/065; A23V 2002/00; A23L 33/105; A61P 3/06

(57) ABSTRACT

A dietary supplement composition, used for treating hypertension in human subjects, includes nicotinamide mononucleotide (NMN) in the amount about 300-600 mg. In another embodiment, the dietary supplement composition includes NMN in the amount about 300-600 mg and trans-resveratrol in the amount about 250-500 mg. A method of treating a hypertension disorder includes the steps of identifying a subject in need of treatment for hypertension, and administering the dietary supplement composition to the subject, in which the dietary supplement composition includes NMN in the amount about 300-600 mg. In another embodiment of method of treating a hypertension disorder includes the steps of identifying a subject in need of treatment of the hypertension, and administering the dietary supplement composition to the subject, in which the dietary supplement composition includes NMN in the amount about 300-600 mg and trans-resveratrol in the amount about 250-500 mg.

### CROSS-REFERENCE TO RELATED APPLICATIONS

The present application is a continuation-in-part of the U.S. application Ser. No. 16/439,201, filed on Jun. 12, 2019. The entire subject matter of this priority document, including specification claims and drawings thereof, is incorporated by reference herein.

### BACKGROUND OF THE INVENTION

### 1. Field of the Invention

The present invention relates to dietary supplement compositions for treating hypertension, and to methods of using the same for treating hypertension in human subjects. More particularly, the present invention relates to dietary supplement compositions which include one of nicotinamide mononucleotide (NMN), and the combination of NMN and trans-resveratrol and to methods of using such dietary supplement compositions for treating hypertension in human subjects.

### 2. Background Art

It is well recognized that hypertension is a major public health concern. It may be noted that hypertension is another term used for high blood pressure. Clinically, hypertension is defined as a systolic blood pressure (SBP) of 140 mm Hg or higher and a diastolic blood pressure (DBP) of 90 mm Hg or higher. It is known that hypertension or high blood pressure without treatment, generally increases the risk of cardiovascular complications such as heart failure, renal failure, aneurysm and heart attack.

In addition, there are a number of collateral effects, including risks for dementia, physical disability and falls/fractures with hypertension patients. In a study, it was found that hypertension causes over 7 million premature deaths per year, and also contributes to 4.5% of the total disease burden worldwide (Reference 1).
Reference 1.
Bramlage P, Hasford J. Blood pressure reduction, persistence and costs in the evaluation of antihypertensive drug treatment—a review. Cardiovascular Diabetology 2009; 8:18-2840-8-18.

Current treatments for hypertension include lifestyle changes as well as drug therapy. The major classes of anti-hypertensive drugs include calcium channel blockers and angiotensin converting enzyme (ACE) inhibitors.

Calcium channel blockers are drugs used to slow the movement of calcium into the cells of the heart and blood vessel walls, which makes it easier for the heart to pump and widens blood vessels. As a result, the heart doesn't have to work as hard, and the patient's blood pressure is lowered. However, some patients suffer the side effects of low blood pressure, e.g., lightheadedness, slower heart rate, drowsiness, constipation, swelling of feet ankles and legs and increased appetite.

ACE inhibitors prevent an enzyme from producing angiotensin II, a substance that narrows the blood vessels. This narrowing of blood vessels can cause high blood pressure and force the heart to work harder. ACE inhibitors prevent the narrowing of blood vessels and help relax veins and arteries to lower the blood pressure. However, some patients taking ACE inhibitors showed cough, itchy skin or rash, salty or metallic taste or a decreased ability to taste, sore throat, swelling of neck and face, etc. ACE inhibitors may cause high potassium levels. This could be a potentially life-threatening complication. Therefore, patients taking ACE inhibitors should regularly have blood tests to measure potassium levels. Although ACE inhibitors help to protect the kidneys, it can also cause kidney failure in some people.

In addition, a significant number of patients that have hypertension are resistant to such drugs. Accordingly, there remains a continuing need for new methods of treating hypertension. The present invention provides one of the answers for treating hypertension.

Accordingly, it is one of the objects of the present invention to provide dietary supplement compositions which overcomes the deficiencies of existing drugs and methods of treating hypertension. The dietary supplement compositions of the present invention include one of nicotinamide mononucleotide (NMN), and the combination of NMN and trans-resveratrol, and to methods of using such dietary supplement compositions for treating hypertension in human subjects.

### SUMMARY OF THE INVENTION

In order to achieve the above objects, the present invention according to a first aspect thereof provides a dietary supplement composition for treating hypertension. The dietary supplement composition according to the first aspect thereof includes nicotinamide mononucleotide (NMN).

The present invention according to a second aspect thereof is characterized in that in the first aspect a content of the NMN is about 300 mg.

The present invention according to a third aspect thereof is characterized in that in the first aspect a content of the NMN is about 300-600 mg.

The dietary supplement composition according to a fourth aspect thereof includes NMN; and trans-resveratrol.

The present invention according to a fifth aspect thereof is characterized in that in the fourth aspect a content of a content of the NMN is about 300-600 mg.

The present invention according to a sixth aspect thereof is characterized in that in the fourth aspect a content of the trans-resveratrol is about 250-500 mg.

The present invention according to a seventh aspect thereof is characterized in that in the fourth aspect a content of the NMN is about 300-600 mg and a content of the trans-resveratrol is about 250-500 mg.

The present invention according to an eighth aspect thereof provides a method of treating a hypertension in a subject. The method includes the steps of identifying a subject in need of treatment of the hypertension; and administering a dietary supplement composition to the subject, the dietary supplement composition including NMN.

The present invention according to a ninth aspect thereof is characterized in that in the eighth aspect a content of the NMN is about 300 mg.

The present invention according to a tenth aspect thereof is characterized in that in the eighth aspect a content of the NMN is about 300-600 mg.

The present invention according to an eleventh aspect thereof provides a method of treating a hypertension in a subject. The method includes the steps of identifying a subject in need of treatment of the hypertension; and administering a dietary supplement composition to the subject, the dietary supplement composition including NMN and trans-resveratrol.

The present invention according to a twelfth aspect thereof is characterized in that in the eleventh aspect a content of the NMN is about 300-600 mg.

The present invention according to a thirteenth aspect thereof is characterized in that in the eleventh aspect a content of the trans-resveratrol is about 250-500 mg.

The present invention according to a fourteenth aspect thereof is characterized in that in the eleventh aspect a content of the NMN is about 300-600 mg and a content of the trans-resveratrol is about 250-500 mg.

The present invention indicates that hypertension is an aging disease and it is modifiable and treatable with the dietary supplements discussed herein. It may be noted that hypertension is another term used for high blood pressure.

The dietary supplements of the present invention use NMN, which successfully treats the subjects with hypertension. It may be noted that NMN is one of most popular dietary supplements for anti-aging as well. Furthermore, it may be noted that NMN activates the longevity gene Sirt1 of blood vessels, and reverses vascular dysfunction and oxidative stress, and hence brings back/lowers the blood pressures to the normal, healthy range.

It has been found that NMN has no effects on the blood pressure within a short time, such as several days or a few weeks. Nonetheless, the applicant of the present invention found that oral administration of the dietary supplement composition including NMN in the amount of 300-600 mg daily can reduce the blood pressure of the patients within a period of three months to a desirable level.

The present invention indicates that hypertension is treatable and/or preventable by daily consumption/intake of the dietary supplement which includes the NMN in the amount of about 300 mg.

The present invention also found that oral administration of the dietary supplement including trans-resveratrol can decrease the systolic blood pressure, but not the diastolic blood pressure. The systolic blood pressure can be decreased within a week by daily administration of the dietary supplement including trans-resveratrol.

In addition, the effects of the dietary supplement that includes NMN on reduction of the blood pressures, can further be helped by daily administering the dietary supplement which further includes trans-resveratrol in the amount of 200-500 mg.

It is recognized that damage to and/or aging of the vascular wall results the loss of flexibility of the blood vessels, leading to the development of hypertension. The dietary supplement which includes a combination of NMN and trans-resveratrol further improves the reduction of blood pressure and brings it back to the normal range.

### DETAILED DESCRIPTION OF EXEMPLARY EMBODIMENTS OF THE INVENTION

Various aspects of the dietary supplement compositions for treating and/or preventing hypertension in human subjects are discussed below. Also discussed are causes of hypertension, concepts for treating hypertension, various formulations of the dietary supplement compositions, studies conducted and results of the studies.

### 1. The Causes of Hypertension

### 1-1. Blood Pressure and Ages

In human subjects, systolic blood pressure (SBP) is the top number/first number on a reading when the heart beats, and diastolic blood pressure (DBP) is the bottom number/second number on a reading when the heart relaxes. In other words, the SBP (the first number) indicates how much pressure a person's blood is exerting against his/her artery walls when his/her heart beats, and the DBP (the second number) indicates how much pressure the person's blood is exerting against his/her artery walls while his/her heart is resting between beats.

The blood pressure SBP/DBP of 3 year old children is about 100/70 mmHg. The blood pressure of 10 year old children is around 110/73 mmHg. The blood pressure increases with age. For example, the blood pressure of most 20 year old youths is 120/80 mmHg approximately. The average blood pressures SBP/DBP of the 30, 40, 50 and 60 year old healthy population are about 122/82, 124/83, 129/85, and 134/88 mmHg respectively.

Obviously, it is recognized that aging causes an increase in blood pressure for humans.

### 1-2. The Hypertension and Aging

The incidence of hypertension is greatest among older adults. Life expectancy continues to increase worldwide, leading to an increase of older adults within the population, which leads to an increase of hypertension patients within the population.

Based on analysis of the data from National Health and Nutrition Examination Survey, spanning 2015-2016 in the United States, it is found that the relevance of hypertension increased with age, from 7.5% among adults aged 18-39 years to 33.2% among those aged 40-59 years, and 63.1% among those aged 60 years and older. A similar pattern was found in both men and women (Reference 2).
Reference 2.
National Center for Health Statistics (NCHS) Data Brief No. 289, October 2017, Page 1-8,

Clearly, based on this it can be concluded that aging generally causes hypertension.

Although the etiology of essential hypertension remains unknown, multiple factors may contribute to the development and pathogenesis of hypertension. Based on the above facts, aging is one of the most important factors that causes hypertension. The present invention explored the treatment of hypertension from the anti-aging perspective, and provides dietary supplements and methods for treating hypertension.

### 2. Two Concepts of Treatment for Hypertension

Hypertension is potentially preventable and/or modifiable. This is based on two concepts: (1) using anti-aging dietary supplement activates the longevity genes, and (2) protecting vascular wall, the endothelial cells. Both blood and lymphatic capillaries are composed of a single layer of the endothelial cells called a monolayer.

### 2-1 Longevity Genes

One of longevity genes in blood vessels is Sirt1, which can be activated by the restriction of calories intake or by administering dietary supplements. One of the most popular anti-aging dietary supplements is nicotinamide mononucleotide (NMN).

A previous research (Reference 3) showed that the dietary=supplement including NMN can activate the Sirt1 gene of blood vessels in mice, which reverses vascular dysfunction and oxidative stress. However, the previous research did not measure the effect of NMN on blood pressure and therefore the effect of administering the dietary supplement including NMN on blood pressure was unknown.
Reference 3.
de Picciotto N E, Gano L B, Johnson L C, Martens C R, Sindler A L, Mills K F, Imai S I, Seals D R. Nicotinamide Mononucleotide Supplementation Reverses Vascular Dysfunction and Oxidative Stress With Aging in Mice. Aging Cell 2016 June; 15(3): 522-530.

A research disclosed in a recent publication (Reference 4) studied the effects of oral administration of NMN within the clinical parameters in healthy Japanese adults. In this research, it was found that NMN is safe. Further, it was found that different dosages of 100 mg, 200 mg and 500 mg of NMN had no effects on the systolic blood pressure and diastolic blood pressure. The study that measured the blood pressure changes within 300 minutes after a single oral administration of each dose of NMN, found that the NMN had no effect on the blood pressure.
Reference 4.
Irie J, Inagaki M, Fujita M, Nakaya H, Mitsuishi M, Yamaguchi S, Yamashita K, Shigaki S. Ono T, Yukioka H, Okano H, Nabeshima Y I, Imai S I, Yasui M, Tsubota K, Itoh H. Effect of Oral Administration Nicotinamide Mononucleotide on Clinical Parameters and Nicotinamide Metabolite Levels in Healthy Japanese Men. Endocrine Journal 2020 Feb. 28; 67(2): 153-160.

According to the research conducted by the applicant of the present invention, NMN has no effects on the blood pressure within a short time period, such as 300 minutes or 3 days. In a study, the applicant of the present invention regularly monitored the blood pressures of patients for a period of six months and found that the blood pressure can be reduced within three months by oral administration of dietary supplement including NMN 300-600 mg daily (details of the study are provided the Examples). Based on the knowledge from public information, the present invention is the first one to show that the dietary supplement including NMN can be used for treatment of hypertension in human subjects.

### 2-2 Endothelial Cells

The flexibility of the blood vessels in a human subject can be improved by administering trans-resveratrol (Reference 5).
Reference 5.
Mattison J A, Wang M, Bernier M, Zhang J, Park S S, Maudsley S, An S S, Santhanam L, Martin B, Faulkner S, Morrell C, Baur J A, Peshkin L, Sosnowska D, Csiszar A, Herbert R L, Tilmont E M, Ungvari Z, Pearson K J, Lakatta E G, de Cabo R. Resveratrol Prevents High Fat/Sucrose Diet-Induced Central Arterial Wall Inflammation and Stiffening in Nonhuman Primates. Cell Metabolism 2014 Jul. 1; 20(1):183-190.

Damage and/or aging of the vascular wall will result in the loss of flexibility of the blood vessels, leading the development of hypertension. The use of trans-resveratrol as discussed in the present invention improves the flexibility of blood vessels, and helps NMN in decreasing the blood pressure.

### 3. Ingredients of Dietary Supplement Compositions

### Part 1. Nicotinamide Mononucleotide (NMN)

NMN is naturally found in fruits and veggies such as avocados, broccoli, cabbage, edamame, and cucumbers. NMN is a direct precursor to the anti-aging NAD+ molecule that is an activator of the longevity genes, such as Sirt1. The chemical structure of NMN is illustrated in the following Illustration 1.

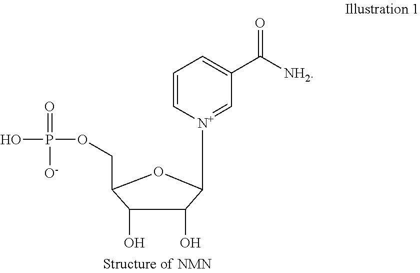 [SMILES: NC(=O)c1ccc[n+](C2OC(COP(=O)([O-])O)C(O)C2O)c1]

The active amount of NMN in a dietary supplement found by the applicant of the present invention is 300 mg to 600 mg per day for treating hypertension in human subjects. In other words, administering the dietary supplement which includes NMN, wherein a content of the NMN is about 300-600 mg, is effective in treating hypertension in human subjects.

### Part 2. Trans-Resveratrol

Trans-resveratrol is one of the primary active ingredients of the dietary supplement of the present invention. Trans-resveratrol is isolated from Japanese knotweed. It has been used for a long time. It is safe for human consumption. The Japanese knotweed, which is also referred to as Reynoutria japonica, synonyms Fallopia japonica and Polygonum cuspidatum, is a large species of herbaceous perennial plant of the knotweed and buckwheat family Polygonaceae.

Chemical structure of trans-resveratrol is illustrated in the following Illustration 2.

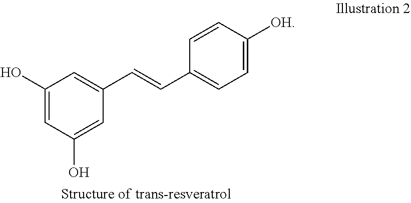 [SMILES: Oc1ccc(/C=C/c2cc(O)cc(O)c2)cc1]

The active amount of trans-resveratrol in a dietary supplement found by the applicant of the present invention is 250 mg to 500 mg per day for treating hypertension in human subjects. In other words, daily administration of the dietary supplement which includes trans-resveratrol, wherein a content of the NMN is about 250 mg to 500 mg, is effective in treating hypertension in human subjects.

### 4. Treatments Using Dietary Supplement Compositions

### 4-1. Formulations

The dietary supplement includes the following Formulas containing one of (1) Part 1—formulations including NMN; (2) Part 2—formulations including trans-resveratrol; and (3) Part 3—formulations including Part 1+Part 2, i.e., NMN and trans-resveratrol. Applicant has conducted more than 9 years of studies to evaluate the efficacy of these dietary supplements for treating hypertension in human subjects.

In the following dietary supplement formulations, a content of each of the active ingredients is specified along with frequency of administering thereof.

Part 1—Formulations including NMN

### Formula N100

The dietary supplement composition according to Formula N100 includes 100 mg of NMN per day.

### Formula N300

The dietary supplement composition according to Formula N300 includes 300 mg of NMN per day.

### Formula N600

The dietary supplement composition according to Formula N600 includes 600 mg of NMN per day.

### Part 2—Formulations Including Trans-Resveratrol

### Formula R200

The dietary supplement composition according to Formula R200 includes 200 mg of trans-resveratrol per day.

### Formula R500

The dietary supplement composition according Formula R500 includes 500 mg of trans-resveratrol per day.

### Part 3—Formulations Including NMN and Trans-Resveratrol

### Formula N300+R200

The dietary supplement compositions according to Formula N300+R200 include 300 mg of NMN and 200 mg of trans-resveratrol per day.

### 4-2. Examples

### Example 1: Treatment of Hypertension Patients with Dietary Supplement Composition/Formula N100, N300, and N600

All adults in this study are primary (SBD<150 mm Hg) hypertension patients who did not take any medication. Patients with systolic blood pressure over 151 mm Hg, who took medications, were excluded.

Twenty-eight hypertension patients aged 48-69 years with SBP 135-150 mm Hg and DBP 89-100 mm Hg, were randomly divided into 4 groups, seven patients in each group. The patients were treated with Formula N100, N300, and N600, or placebo multivitamins daily.

### Results

### Formula N100 Group

The average systolic blood pressure decreased by about 12 mmHg, the diastolic blood pressure decreased by about 5 mm Hg after a period of six months of administering N100 formula daily. Four of seven patients showed no effects. This group shows little effect.

### Formula N300 Group

In this group, the average systolic blood pressure decreased by 18-26 mmHg, and the diastolic blood pressure decreased by 10-17 mm Hg after a period of six months of administering N300 formula daily. Both systolic blood pressure and diastolic blood pressure decreased to the normal and healthy range. The results of the study from this group indicates that hypertension is treatable and/or modifiable by taking a dietary supplement composition according to Formula N300 which includes 300 mg of NMN per day. The response rate is about half of the patients in this trial group. Three of seven patients showed no effect.

### Formula N600 Group

In this group, the average systolic blood pressure decreased by 14-27 mmHg, the diastolic blood pressure decreased by 10-15 mm Hg after a period of six months of administering the N600 formula daily. Both systolic blood pressure and diastolic blood pressure decreased to the normal healthy range. It indicates hypertension is modifiable by taking the dietary supplement which includes NMN 600 mg daily. Four of the seven patients showed no effect.

### The Placebo Group

All patients who were administered the placebo showed no effect from the placebo on their systolic blood pressure and diastolic blood pressure.

### Example 2: Treatment of Hypertension Patients with Formula R200 and R500

Twenty-one hypertension patients aged 48-69 with SBP 135-150 mm Hg and DBP 89-100 mm Hg, were randomly divided to 3 groups, seven patients in each group. The patients were treated with Formula R200, R500, and placebo multivitamins daily.

### Results

Trans-resveratrol is a fast-acting compound. After administering a dietary supplement composition including trans-resveratrol daily, blood pressures of the patients were measured every day for the first week, then measured weekly.

### Formula R200 Group

In this group, each subject was administered with Formula R200 daily, i.e., each subjected ingested 200 mg of trans-resveratrol per day.

There were no changes in average systolic blood pressure and diastolic blood pressure for the first 3 days. The average systolic blood pressure decreased by about 10 to 15 mmHg after 7 days for the group who had taken 200 mg trans-resveratrol daily. But the diastolic blood pressure had no change. When observed, the systolic blood pressure did not decrease after two weeks. Two of seven patients showed no effect at all. The response rate of decreasing systolic blood pressure was 5/7.

### Formula R500 Group

In this group, no changes were recorded in the average systolic blood pressure and diastolic blood pressure within 3 days of administering the R500 formula. The systolic blood pressure decreased by about 15 mm Hg after daily administration of the dietary supplement that includes 500 mg trans-resveratrol for a period of 7 days. The diastolic blood pressure remained the same with no change. When observed, the systolic blood pressure did not decrease after week two for the administration of the R500 formula. One of seven patients showed no effect at all.

### The Placebo Group

All patients showed no effect from the placebo.

### Example 3: Treatment of Hypertension Patients with Formula N300+R200

Fifteen hypertension patients aged 45-67 with SBP 135-150 mm Hg and DBP 89-100 mm Hg, were treated with Formula N300+R200, i.e., were administered NMN 300 mg and trans-resveratrol 200 mg daily. Another group of seven hypertension patients was treated with placebo multivitamins daily.

### Results

The measurements of blood pressure was performed weekly for the first month, then monthly.

The average systolic blood pressure decreased by about 10 mm Hg, and the diastolic blood pressure decreased by about 5 mm Hg after one week of administering Formula N300+R200 daily. The response rate is 11/15.

The average systolic blood pressure decreased by about 16 mm Hg, and the diastolic blood pressure decreased by about 8 mm Hg after three weeks from the beginning of the treatment. The response rate is 11/15.

The average systolic blood pressure decreased by about 18 mm Hg, and the diastolic blood pressure decreased about 8 mm Hg after one month from the beginning of the treatment. The response rate is 11/15.

The average systolic blood pressure decreased by about 15-21 mm Hg, and the diastolic blood pressure decreased about 5-10 mm Hg after two months from the beginning of the treatment. The response rate increased to 12/15.

The systolic blood pressure decreased by about 15-28 mm Hg, the diastolic blood pressure decreased by about 7-15 mm Hg after three and six months from the beginning of the treatment. The response rate remains 12/15.

### The Placebo Group

All patients showed no effects.

### Example 4: Prevention of Hypertension with Formula N300

Ten healthy adults aged 50-60 with body mass index (BMI) between 20-25, SBP 118-125 mm Hg and DBP 75-85 mm Hg, were treated with NMN 300 mg daily for two years. Twenty healthy adults were treated with placebo multivitamins daily.

### Results

The measurements for blood pressure were performed every 6 months. It showed that the average systolic blood pressure slightly decreased by about 2 to 3 mm Hg for the adults who took the dietary supplement containing NMN 300 mg daily, and there was no significant change in diastolic blood pressure during two years. Some participants reported that they were not prone to catch colds in winter time when they usually had it.

The placebo group with multivitamins showed that their BMI increased from about 20-25 to about 20-27. One of them, the systolic blood pressure increased by about 18 mm Hg, and diastolic blood pressure increased by about 6 mm Hg. Four adults dropped out from this group during the first 2 years.

Although the present invention has been described herein with respect to a number of specific illustrative embodiments, the foregoing description is intended to illustrate, rather than to limit the invention. Those skilled in the art will realize that many modifications of the illustrative embodiment could be made which would be operable. All such modifications, which are within the scope of the claims, are intended to be within the scope and spirit of the present invention.dsf

What is claimed is:

1. A dietary supplement composition comprising:
nicotinamide mononucleotide (NMN).

2. The dietary supplement composition according to claim 1, wherein a content of said NMN is about 300 mg.

3. The dietary supplement composition according to claim 1, wherein a content of said NMN is about 300-600 mg.

4. A dietary supplement composition comprising:
nicotinamide mononucleotide (NMN); and
trans-resveratrol.

5. The dietary supplement composition according to claim 4, wherein a content of said NMN is about 300-600 mg.

6. The dietary supplement composition according to claim 4, wherein a content of the trans-resveratrol is about 250-500 mg.

7. The dietary supplement composition according to claim 4, wherein a content of said NMN is about 300-600 mg and a content of said trans-resveratrol is about 250-500 mg.

8. A method of treating a hypertension in a subject, said method comprising the steps of:
selecting a subject in need of treatment of the hypertension, and
administering a dietary supplement composition to said subject; said dietary supplement composition comprising nicotinamide mononucleotide (NMN).

9. The method of treating a hypertension disorder in a subject according to claim 8, wherein a content of said NMN is about 300 mg.

10. The method of treating a hypertension disorder in a subject according to claim 8, wherein a content of said NMN is about 300-600 mg.

11. A method of treating a hypertension disorder in a subject, said method comprising the steps of:
selecting a subject in need of a treatment of the hypertension disorder, and
administering a dietary supplement composition to said subject; said dietary supplement composition comprising nicotinamide mononucleotide (NMN) and trans-resveratrol.

12. The method of treating a hypertension disorder in a subject according to claim 11, wherein a content of said NMN is about 300-600 mg.

13. The method of treating a hypertension disorder in a subject according to claim 11, wherein a content of the trans-resveratrol is about 250-500 mg.

14. The method of treating a hypertension disorder in a subject according to claim 11, wherein a content of said NMN is about 300-600 mg and a content of said trans-resveratrol is about 250-500 mg.
